(19) United States
(12) Patent Application Publication
(10) Pub. No.: US 2025/0143826 A1
(43) Pub. Date: May 8, 2025

(54) HOUSING FOR A STERILE COVER AND A FILTER

(71) Applicant: Mazor Robotics Ltd., Caesarea (IL)

(72) Inventor: Zhinan Guo, Shanghai (CN)

(21) Appl. No.: 18/835,992

(22) PCT Filed: Feb. 7, 2022
(86) PCT No.: PCT/CN2022/075392

§ 371 Date: Aug. 5, 2024

(51) Int. Cl. A61B 46/10; A61B 34/30; A61B 90/00; G03B 11/00

(52) U.S. Cl. CPC A61B 46/10; A61B 34/30; A61B 90/361; G03B 11/00

(57) ABSTRACT

A drape assembly (100) for a surgical instrument (108) is provided. The assembly (100) comprises a covering (106) having an outer perimeter and an opening (118) defining an inner perimeter. The covering (106) is configured to cover an instrument (108). The assembly (100) also comprises a housing (102) positioned within the opening (118) and connected to the inner perimeter. The housing (102) is configured to removably couple to the instrument (108). The assembly (100) also comprises an optical element (104) positioned within the housing (102).

### BACKGROUND

The present disclosure is generally directed to housings, and relates more particularly housings for coupling a sterile cover to a surgical instrument.

Surgical sites typically require a sterile environment. Surgical instruments and/or robots may be used in such sterile environments. Maintaining the sterility of robots and surgical instruments is particularly important.

### BRIEF SUMMARY

Example aspects of the present disclosure include:

A drape assembly for a surgical instrument according to at least one embodiment of the present disclosure comprises a covering having an outer perimeter and an opening defining an inner perimeter, the covering configured to cover an instrument; a housing positioned within the opening and connected to the inner perimeter, the housing configured to removably couple to the instrument; and an optical element positioned within the housing.

Any of the aspects herein, wherein the housing comprises an outer surface, an inner surface, a first end, a second end opposite the first end, a first groove positioned on the outer surface between the first end and the second end, and a second groove positioned on the inner surface between the first end and the second end.

Any of the aspects herein, wherein the first groove receives the inner perimeter of the covering and a first ring, the first ring configured to secure the inner perimeter to the first groove, thereby connecting the housing to the inner perimeter, and wherein the second groove receives a second ring configured to secure the optical element within the housing.

Any of the aspects herein, wherein each of the first ring and the second ring is at least one of a c-spring, a band, or a cable tie.

Any of the aspects herein, wherein the second end of the housing is threaded and configured to thread into a threaded receiver of the instrument.

Any of the aspects herein, wherein the instrument is a camera disposed at an end of an arm, wherein the covering is configured to cover the camera and the arm.

Any of the aspects herein, wherein the inner perimeter is connected to the housing via adhesion.

Any of the aspects herein, wherein the optical element is a UV filter.

A housing configured to couple a surgical instrument and a covering according to at least one embodiment of the present disclosure comprises a body having an outer surface and an opening defining an inner surface; a first securement feature disposed on the outer surface and configured to secure the covering to the body; and a second securement feature disposed on the inner surface and configured to secure an optical element in the opening such that the optical element has a predetermined position relative to the covering.

Any of the aspects herein, wherein the body comprises a first surface opposite a second surface, the first surface and the second surface perpendicular to the outer surface, wherein the body further comprises a receiving surface inset from the first surface, the receiving surface configured to receive the optical element, the receiving surface having a diameter less than a diameter of the outer surface, and wherein the second securement feature is between the receiving surface and the first surface.

Any of the aspects herein, wherein the body comprises a threaded protrusion extending from the second surface, the threaded protrusion having a diameter less than a diameter of the outer surface, wherein the opening extends through the threaded protrusion, and wherein the threaded protrusion is configured to thread into a threaded receiver of an instrument.

Any of the aspects herein, wherein the first securement feature extends around a perimeter of the outer surface.

Any of the aspects herein, wherein the second securement feature extends around a perimeter of the inner surface.

Any of the aspects herein, wherein the body is annular.

An assembly for a surgical instrument according to at least one embodiment of the present disclosure comprises a covering having an outer perimeter and an opening defining an inner perimeter, the covering configured to cover an instrument; a housing comprising: a body having an outer surface and an opening defining an inner surface, the inner surface and the outer surface extending from a first end to a second end; a first securement feature configured to secure the covering to the housing; a second securement feature configured to secure an optical element to the housing; and the optical element positioned within the opening of the housing.

Any of the aspects herein, wherein the first securement feature comprises a first groove disposed on the outer surface and configured to receive a first ring and the second securement feature comprises a second groove disposed on the inner surface and configured to receive a second ring, each of the first ring and the second ring is at least one of a c-spring, a band, or a cable tie.

Any of the aspects herein, wherein the second end of the housing is threaded and configured to thread into a threaded receiver of the instrument.

Any of the aspects herein, wherein the instrument is a camera disposed at an end of an arm, wherein the covering is configured to cover the camera and the arm.

Any of the aspects herein, wherein the inner perimeter is connected to the housing via adhesion.

Any of the aspects herein, wherein the optical element is a UV filter.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view of a housing and a filter housed in the housing according to at least one embodiment of the present disclosure;

FIG. 1B is a perspective view of the housing shown in FIG. 1A without the filter according to at least one embodiment of the present disclosure;

FIG. 1C is a top view of the housing shown in FIG. 1B;

FIG. 2A is a side view of a housing according to at least one embodiment of the present disclosure;

FIG. 2B is a cross section side view of the housing shown in FIG. 1C according to at least one embodiment of the present disclosure;

FIG. 3A is a side view of an assembly according to at least one embodiment of the present disclosure;

FIG. 3B is a side exploded view of an assembly according to at least one embodiment of the present disclosure;

FIG. 4 is a perspective view of a housing, a drape, and a surgical instrument disposed on an arm according to at least one embodiment of the present disclosure;

FIG. 5 is a block diagram of a system according to at least one embodiment of the present disclosure; and

FIG. 6 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

The present disclosure relates generally to a sterile drape or covering design for covering operating room equipment and, more specifically, to a disposable sterile drape or covering for a surgical instrument such as, for example, an field camera.

Typically, a field camera used in an operating room includes an autozoom camera and a mechanical arm fixed at a top of a shadowless lamp above an operation table. Since the field camera is just above the operation table, the field camera is positioned in a sterile environment. When using conventional drapes, the whole field camera is covered by a disposable sterile drape or covering, resulting in a decrease in quality of the field camera video resolution through the disposable sterile drape or covering. The sterile drape or covering's light transmittance is very low, less than 80% and the drape is not flat around the camera lens area which may also degrade the video resolution. In instances where a sterile environment is not maintained, a UV filer may be attached to the front of the camera lens to protect the camera lens and its light transmittance can be above 99%.

At least one embodiment of the present disclosure provides for an integrated sterile drape with a UV filter. A housing for holding the UV filter features a circular groove feature to attach and/or seal the disposable sterile drape or covering. The attachment and/or sealing may include, for example, a C-type spring, an over mold, adhesive, or the like.

Embodiments of the present disclosure provide technical solutions to one or more of the problems of (1) providing a sterile boundary between a sterile environment and a non-sterile instrument, (2) maintaining a resolution quality of a camera while forming a sterile boundary between the camera and a sterile environment, (3) providing a housing for removably coupling a cover and an optical element to a surgical instrument, and (4) providing a housing in which a cover and an optical element are removably secured to the housing.

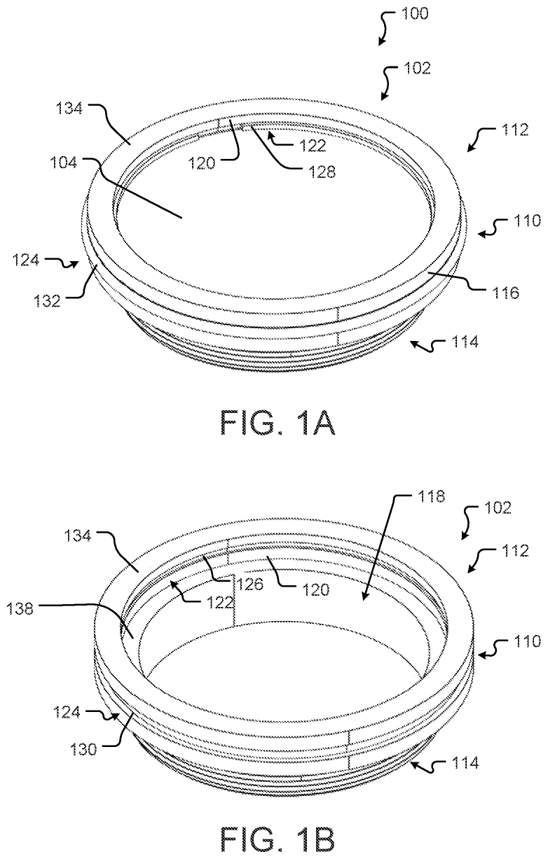

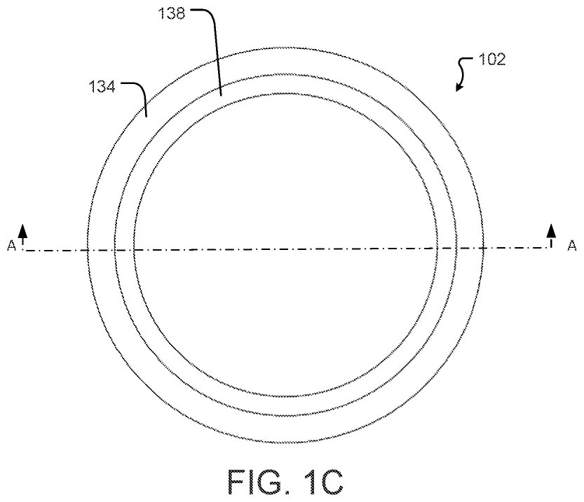

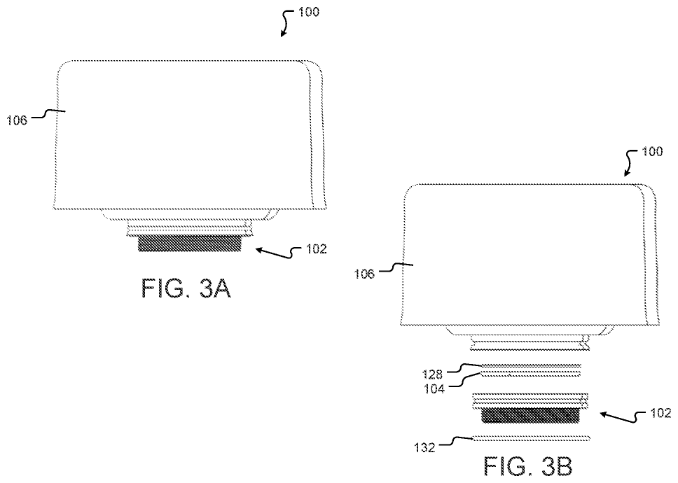

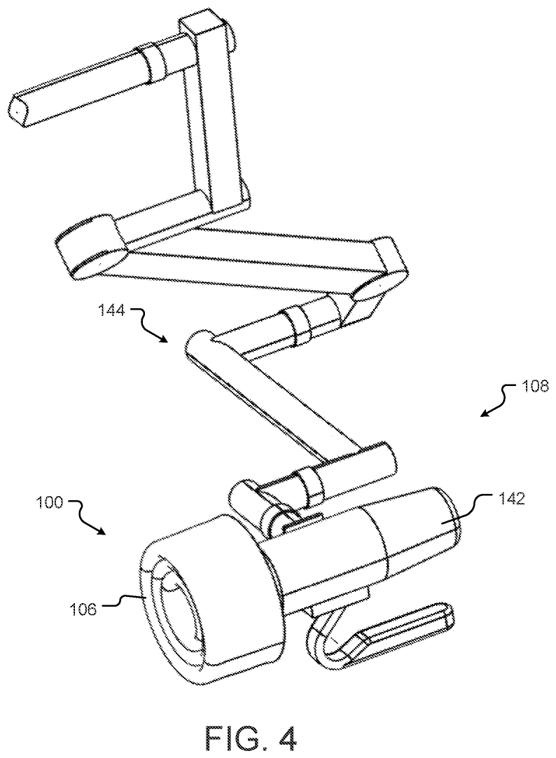

With reference now to FIGS. 1A-4, an assembly 100 is shown and will be described in further detail. In some embodiments, the assembly 100 is referred to as a “drape assembly” 100. The assembly 100 is configured to cover or drape a surgical instrument 108 (shown in FIG. 4) to form a barrier or boundary between the surgical instrument 108 and an environment. In some embodiments, the assembly 100 comprises a housing 102 for housing an optical element 104 and for coupling a drape or covering 106 (shown in FIGS. 3A, 3B, and 4) to the surgical instrument 108. The housing 102 comprises a body 110 extending from a first end 112 to a second end 114. The body 110 also comprises an outer surface 116, an opening 118, and an inner surface 120. As shown in the illustrated embodiment, the body 110 is annular, though in the other embodiments the body 110 may be any shape.

The housing 102 comprises a first securement feature 122 configured to secure the drape or covering 106 to the housing 102 and a second securement feature 124 configured to secure the optical element 104 to the housing 102. More specifically, the second securement feature 124 is configured to secure the optical element 104 in the opening 118 of the housing 102 such that the optical element 104 has a predetermined position relative to the drape or covering 106.

In the illustrated embodiment, the first securement feature 122 comprises a first groove 126 (visible in FIG. 1B) disposed on the outer surface 116 of the body 110 between the first end 112 and the second end 114. In the illustrated embodiment, the first groove 126 extends around an entire perimeter of the outer surface 116, though in other embodiments the first groove 126 may extend around a portion of the perimeter. The first groove 126 is configured to receive the drape or covering 106 and a first ring 128 for securing the drape or covering 106 to the first groove 126. More specifically, the drape or covering 106 may comprise an outer perimeter and an opening defining an inner perimeter. The opening of the housing 102 and the opening of the drape or cover 106 may be aligned and the inner perimeter of the drape or covering 106 may be secured to the first groove 126 by the first ring 128. The first ring 128 may be, for example, a C-ring, a snap ring, a cable tie, a band, or the like.

It will be appreciated that in other embodiments, the first securement feature 122 may comprise adhesion. In other words, the drape or covering 106 may be adhered to the housing 102. In such embodiments, the housing 102 may or may not include the first groove 126 and the drape or covering 106 may be adhered to the first groove 126 or any portion of the outer surface 116. The first securement feature 122 provides for easy attachment and/or removal of the drape or covering 106 to the housing 102.

In some instances, the drape or covering 106 may be replaced after use and may be simple removed from the housing 102 and another drape or covering 106 may be attached to the housing 102. For example, in embodiments where the first securement feature 122 comprises the first groove 126 and the first ring 128, the first ring 128 may be removed from the first groove 126, a first drape or covering 106 may be removed from the first groove 126, a second drape or covering 106 may be positioned at the first groove 126, and the first ring 128 (whether the same first ring 128 or a different first ring 128) may secure the second drape or covering 106 to the first groove 126. Thus, the drape or covering 106 may be easily removed and/or replaced from the housing 102.

As illustrated, the optical element 104 is positioned in the opening 118 and secured by the second securement feature 124. The optical element 104 may be or include, for example, a lens, a filter (e.g., a UV filter), a mirror, a cover, combinations thereof, or the like. The optical element 104 may be, in some embodiments, sterilized.

Turning to FIG. 1B, in the illustrated embodiment, the second securement feature 124 comprises a second grove 130 disposed on the inner surface 120 of the body 110 between the first end 112 and the second end 114. In the illustrated embodiment, the second grove 130 extends around an entire perimeter of the inner surface 120, though in other embodiments the second grove 130 may extend around a portion of the perimeter. The second grove 130 is configured to receive a second ring 132 (visible in FIG. 1A) for securing the optical element 104 to the housing 102. More specifically, the body 110 may comprise a first surface 134 at the first end 112 opposite a second surface 136 at the second end 114. The first surface and the second surface 136 may be perpendicular to the outer surface 116. The body 110 further comprises a receiving surface 138 inset from the first surface 134. The receiving surface 138 is configured to receive the optical element 104 and combined with the second ring 132, secures the optical element 104 in the housing 102. In other words, the optical element 104 is positioned and held between the receiving surface 138 and the second ring 132. The receiving surface 138 comprises a diameter less than a diameter of the outer surface. As shown in the illustrated embodiment, the second grove 130 is positioned between the receiving surface 138 and the first surface 134.

It will be appreciated that in other embodiments, the second securement feature 124 may comprise adhesion. In other words, the optical element 104 may be adhered to the housing 102. In such embodiments, the housing 102 may or may not include the second grove 130 and the optical element 104 may be adhered to the inner surface 120 and/or the receiving surface 138.

In some instances, the optical element 104 may be replaced or sterilized after use and may be simple removed from the housing 102 for replacement or sterilization. For example, in embodiments where the second securement feature 124 comprises the second grove 130 and the second ring 132, the second ring 132 may be removed from the second grove 130, and the optical element 104 may be removed from the opening 118 of the housing 102. The optical element 104 may be replaced or sterilized for reuse. Thus, the optical element 104 may be easily removed and/or replaced from the housing 102 for any reason.

FIG. 1C is a top view of the housing 102, illustrating the first surface 134 and the receiving surface 138.

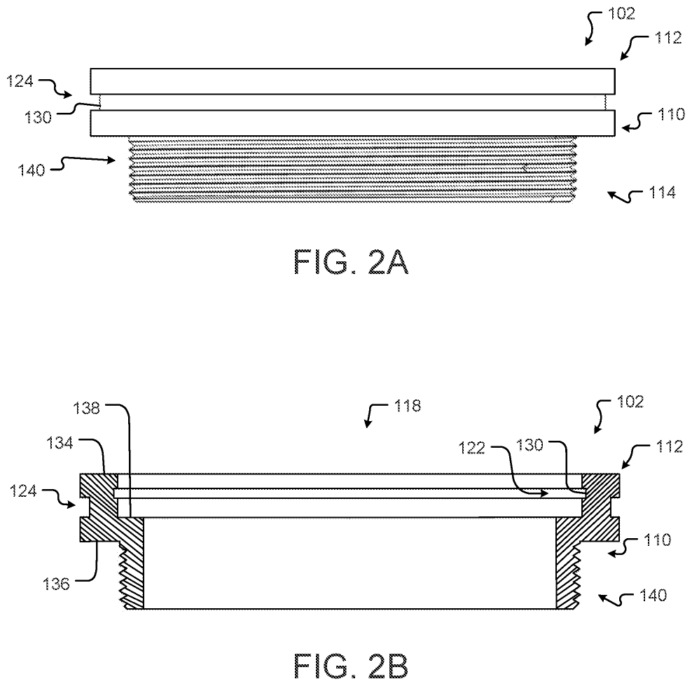

Turning to FIGS. 2A and 2B, a side view of the housing 102 and a cross-section view of the housing 102 taken from A-A in FIG. 1C are respectively shown. The body 110 comprises a protrusion 140 extending from the second surface. In the illustrated embodiment, the protrusion 140 is threaded, though it will be appreciated that in other embodiments, the protrusion 140 may not be threaded. The protrusion 140 comprises a diameter less than a diameter of the outer surface 116 and the opening 118 of the body 110 extends through the protrusion 140. The protrusion 140 is configured to be received by a receiver of the surgical instrument 108. In embodiments where the protrusion 140 is threaded, the receiver comprises a bore having an inner threaded surface for receiving the threaded protrusion 140.

Turning to FIGS. 3A and 3B, a side view of the assembly 100 and an exploded side view of the assembly 100 are shown, respectively. As previously described, the drape or covering 106 is attached to the housing 102 via the first securement feature 122. In the illustrated embodiment, the drape or covering 106 is secured to the first groove 126 by the first ring 128. The assembly 100 is shown in a shipping configuration, in which the drape or covering 106 is folded in front of the housing 102. In some embodiments, the drape or covering 106 may be coupled to the housing 102 at a first location (e.g., a manufacturer) and shipped to a second location (e.g., a surgical site) in the shipping configuration. It will be appreciated that in other embodiments, the drape or covering 106, the housing 102, and the first securement feature 122 may be shipped as separate components and assembled on site.

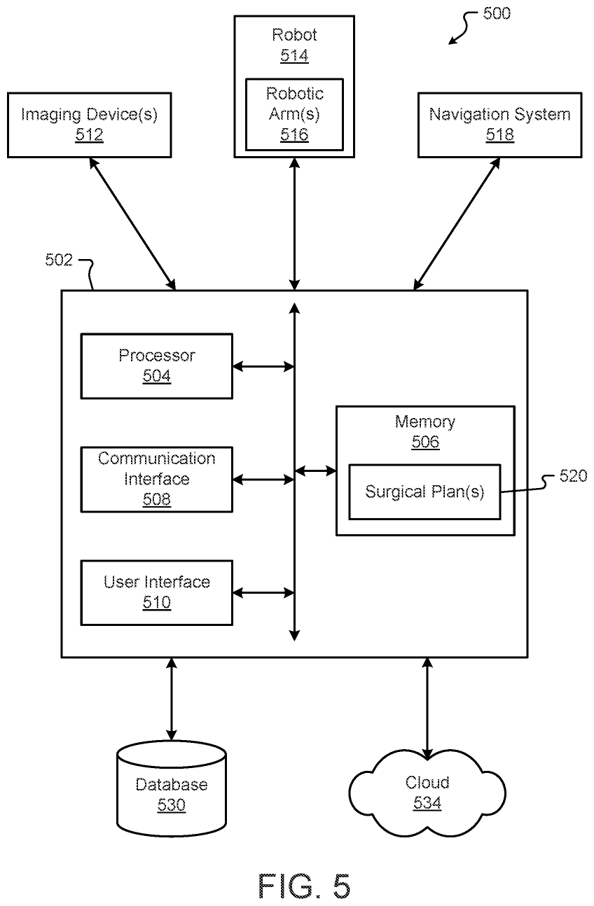

Turning to FIG. 4, the assembly 100 and the surgical instrument 108 are shown. The assembly 100 may be moved to a working configuration in which the housing 102 is coupled to the surgical instrument 108. The drape or covering 106 may be unfolded and draped over the surgical instrument 108, thereby forming a boundary between the surgical instrument 108 and an environment. In some embodiments, the drape or covering 106 may form a sterile boundary between the surgical instrument 108, which may be unsterile, and a sterile environment, such as a surgical site. In the illustrated embodiment, the surgical instrument 108 comprises a camera 142 and an arm 144. The arm 144 may be, for example, a robotic arm 516 (described with respect to FIG. 5). The camera 142 may be, for example, an imaging device 512 (also described with respect to FIG. 5). In such embodiments, the drape or covering 106 is configured to cover the camera 142 and the arm 144. The drape or covering 106 may be removed and replaced after use, though it will be appreciated that in some embodiments, the drape or covering 106 may be reused and/or sterilized before reuse. The optical element 104 may be reused after use. In some embodiments, the optical element 104 may be sterilized after use, then reused. It will be appreciated that in other embodiments, the optical element 104 may be replaced.

The assembly 100 beneficially provides for a reusable housing for coupling a drape or covering and an optical element to a, for example, a camera to form a sterile boundary between the camera and a sterile environment, while also maintaining a quality of a resolution of the camera. In embodiments where the optical element 104 is a UV filter, the UV filter provides a clear, sterile boundary between a lens of the camera and the sterile environment without deteriorating the quality of the resolution of the camera. Further, the assembly 100 is easy and quick to use, and provides for simple replacement of parts (e.g., the optical element 104 and/or the drape or covering 106).

Turning to FIG. 5, a block diagram of a system 500 according to at least one embodiment of the present disclosure is shown. The system 500 may be used to control, pose, or otherwise operate a surgical instrument (e.g., a camera and an arm) and/or carry out one or more other aspects of one or more of the methods disclosed herein. The system 500 comprises a computing device 502, one or more imaging devices 512, a robot 514, a navigation system 518, a database 530, and/or a cloud or other network 534. Systems according to other embodiments of the present disclosure may comprise more or fewer components than the system 500. For example, the system 500 may not include the imaging device 512, the robot 514, the navigation system 518, one or more components of the computing device 502, the database 530, and/or the cloud 534.

The computing device 502 comprises a processor 504, a memory 506, a communication interface 508, and a user interface 510. Computing devices according to other embodiments of the present disclosure may comprise more or fewer components than the computing device 502.

The processor 504 of the computing device 502 may be any processor described herein or any similar processor. The processor 504 may be configured to execute instructions stored in the memory 506, which instructions may cause the processor 504 to carry out one or more computing steps utilizing or based on data received from the imaging device 512, the robot 514, the navigation system 518, the database 530, and/or the cloud 534.

The memory 506 may be or comprise RAM, DRAM, SDRAM, other solid-state memory, any memory described herein, or any other tangible, non-transitory memory for storing computer-readable data and/or instructions. The memory 506 may store one or more surgical plan(s) 520, which may comprise one or more steps for completing a surgical procedure. The memory 506 may store information or data useful for completing, for example, any step of the method 600 described herein, or of any other methods. The memory 506 may store, for example, instructions and/or machine learning models that support one or more functions of the robot 514. For instance, the memory 506 may store content (e.g., instructions and/or machine learning models) that, when executed by the processor 504, enable one or more processes. Such content, if provided as in instruction, may, in some embodiments, be organized into one or more applications, modules, packages, layers, or engines. Alternatively or additionally, the memory 506 may store other types of content or data (e.g., machine learning models, artificial neural networks, deep neural networks, etc.) that can be processed by the processor 504 to carry out the various method and features described herein. Thus, although various contents of memory 506 may be described as instructions, it should be appreciated that functionality described herein can be achieved through use of instructions, algorithms, and/or machine learning models. The data, algorithms, and/or instructions may cause the processor 504 to manipulate data stored in the memory 506 and/or received from or via the imaging device 512, the robot 514, the database 530, and/or the cloud 534.

The computing device 502 may also comprise a communication interface 508. The communication interface 508 may be used for receiving image data or other information from an external source (such as the imaging device 512, the robot 514, the navigation system 518, the database 530, the cloud 534, and/or any other system or component not part of the system 500), and/or for transmitting instructions, images, or other information to an external system or device (e.g., another computing device 502, the imaging device 512, the robot 514, the navigation system 518, the database 530, the cloud 534, and/or any other system or component not part of the system 500). The communication interface 508 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 508 may be useful for enabling the device 502 to communicate with one or more other processors 504 or computing devices 502, whether to reduce the time needed to accomplish a computing-intensive task or for any other reason.

The computing device 502 may also comprise one or more user interfaces 510. The user interface 510 may be or comprise a keyboard, mouse, trackball, monitor, television, screen, touchscreen, and/or any other device for receiving information from a user and/or for providing information to a user. The user interface 510 may be used, for example, to receive a user selection or other user input regarding any step of any method described herein. Notwithstanding the foregoing, any required input for any step of any method described herein may be generated automatically by the system 500 (e.g., by the processor 504 or another component of the system 500) or received by the system 500 from a source external to the system 500. In some embodiments, the user interface 510 may be useful to allow a surgeon or other user to modify instructions to be executed by the processor 504 according to one or more embodiments of the present disclosure, and/or to modify or adjust a setting of other information displayed on the user interface 510 or corresponding thereto.

Although the user interface 510 is shown as part of the computing device 502, in some embodiments, the computing device 502 may utilize a user interface 510 that is housed separately from one or more remaining components of the computing device 502. In some embodiments, the user interface 510 may be located proximate one or more other components of the computing device 502, while in other embodiments, the user interface 510 may be located remotely from one or more other components of the computer device 502.

The imaging device 512 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 512, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 512 may be used to obtain first image data (e.g., a first image) at a first time, and a second imaging device 512 may be used to obtain second image data (e.g., a second image) at a second time after the first time. The imaging device 512 may be capable of taking a 2D image or a 3D image to yield the image data. The imaging device 512 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 512 suitable for obtaining images of an anatomical feature of a patient. The imaging device 512 may be contained entirely within a single housing, or may comprise a transmitter/emitter and a receiver/detector that are in separate housings or are otherwise physically separated.

In some embodiments, the imaging device 512 may comprise more than one imaging device 512. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 512 may be operable to generate a stream of image data. For example, the imaging device 512 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

In some embodiments, the assembly 100 may be installed on the imaging device 512 such that the housing 102 is secured to the imaging device 512 and the drape or covering 106 covers the imaging device 512.

The robot 514 may be any surgical robot or surgical robotic system. The robot 514 may be or comprise, for example, the Mazor X™ Stealth Edition robotic guidance system. The robot 514 may be configured to position the imaging device 512 at one or more precise position(s) and orientation(s), and/or to return the imaging device 512 to the same position(s) and orientation(s) at a later point in time. The robot 514 may additionally or alternatively be configured to manipulate a surgical tool (whether based on guidance from the navigation system 518 or not) to accomplish or to assist with a surgical task. In some embodiments, the robot 514 may be configured to hold and/or manipulate an anatomical element during or in connection with a surgical procedure. The robot 514 may comprise one or more robotic arms 516. In some embodiments, the robotic arm 516 may comprise a first robotic arm and a second robotic arm, though the robot 514 may comprise more than two robotic arms. In some embodiments, one or more of the robotic arms 516 may be used to hold and/or maneuver the imaging device 512. In embodiments where the imaging device 512 comprises two or more physically separate components (e.g., a transmitter and receiver), one robotic arm 516 may hold one such component, and another robotic arm 516 may hold another such component. Each robotic arm 516 may be positionable independently of the other robotic arm. The robotic arms 516 may be controlled in a single, shared coordinate space, or in separate coordinate spaces.

The robot 514, together with the robotic arm 516, may have, for example, one, two, three, four, five, six, seven, or more degrees of freedom. Further, the robotic arm 516 may be positioned or positionable in any pose, plane, and/or focal point. The pose includes a position and an orientation. As a result, an imaging device 512, surgical tool, or other object held by the robot 514 (or, more specifically, by the robotic arm 516) may be precisely positionable in one or more needed and specific positions and orientations.

The robotic arm(s) 516 may comprise one or more sensors that enable the processor 504 (or a processor of the robot 514) to determine a precise pose in space of the robotic arm (as well as any object or element held by or secured to the robotic arm).

In some embodiments, reference markers (e.g., navigation markers) may be placed on the robot 514 (including, e.g., on the robotic arm 516), the imaging device 512, or any other object in the surgical space. The reference markers may be tracked by the navigation system 518, and the results of the tracking may be used by the robot 514 and/or by an operator of the system 500 or any component thereof. In some embodiments, the navigation system 518 can be used to track other components of the system (e.g., imaging device 512) and the system can operate without the use of the robot 514 (e.g., with the surgeon manually manipulating the imaging device 512 and/or one or more surgical tools, based on information and/or instructions generated by the navigation system 518, for example).

The navigation system 518 may provide navigation for a surgeon and/or a surgical robot during an operation. The navigation system 518 may be any now-known or future-developed navigation system, including, for example, the Medtronic StealthStation™ S8 surgical navigation system or any successor thereof. The navigation system 518 may include one or more cameras or other sensor(s) for tracking one or more reference markers, navigated trackers, or other objects within the operating room or other room in which some or all of the system 500 is located. The one or more cameras may be optical cameras, infrared cameras, or other cameras. In some embodiments, the navigation system 518 may comprise one or more electromagnetic sensors. In various embodiments, the navigation system 518 may be used to track a position and orientation (e.g., a pose) of the imaging device 512, the robot 514 and/or robotic arm 516, and/or one or more surgical tools (or, more particularly, to track a pose of a navigated tracker attached, directly or indirectly, in fixed relation to the one or more of the foregoing). The navigation system 518 may include a display for displaying one or more images from an external source (e.g., the computing device 502, imaging device 512, or other source) or for displaying an image and/or video stream from the one or more cameras or other sensors of the navigation system 518. In some embodiments, the system 500 can operate without the use of the navigation system 518. The navigation system 518 may be configured to provide guidance to a surgeon or other user of the system 500 or a component thereof, to the robot 514, or to any other element of the system 500 regarding, for example, a pose of one or more anatomical elements, whether or not a tool is in the proper trajectory, and/or how to move a tool into the proper trajectory to carry out a surgical task according to a preoperative or other surgical plan.

The database 530 may store information that correlates one coordinate system to another (e.g., one or more robotic coordinate systems to a patient coordinate system and/or to a navigation coordinate system). The database 530 may additionally or alternatively store, for example, one or more surgical plans (including, for example, pose information about a target and/or image information about a patient's anatomy at and/or proximate the surgical site, for use by the robot 514, the navigation system 518, and/or a user of the computing device 502 or of the system 500); one or more images useful in connection with a surgery to be completed by or with the assistance of one or more other components of the system 500; and/or any other useful information. The database 530 may be configured to provide any such information to the computing device 502 or to any other device of the system 500 or external to the system 500, whether directly or via the cloud 534. In some embodiments, the database 530 may be or comprise part of a hospital image storage system, such as a picture archiving and communication system (PACS), a health information system (HIS), and/or another system for collecting, storing, managing, and/or transmitting electronic medical records including image data.

The cloud 534 may be or represent the Internet or any other wide area network. The computing device 502 may be connected to the cloud 534 via the communication interface 508, using a wired connection, a wireless connection, or both. In some embodiments, the computing device 502 may communicate with the database 530 and/or an external device (e.g., a computing device) via the cloud 534.

The system 500 or similar systems may be used, for example, to carry out one or more aspects of any of the method 600 described herein. The system 500 or similar systems may also be used for other purposes.

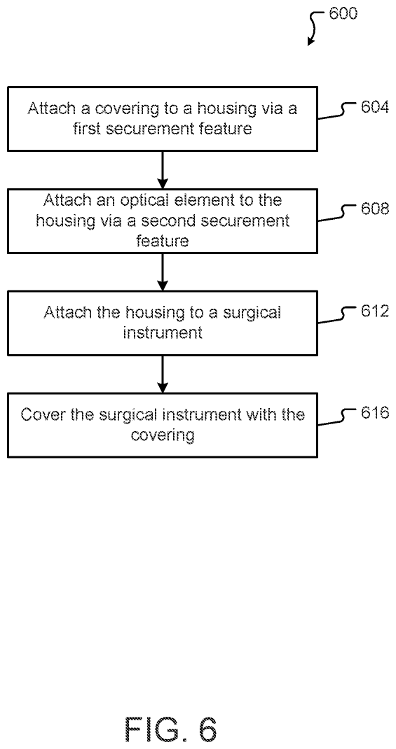

FIG. 6 depicts a method 600 that may be used, for example, for assembling and installing an assembly comprising a housing configured to receive a cover and an optical element.

The method 600 (and/or one or more steps thereof) may be carried out or otherwise performed, for example, by at least one processor. The at least one processor may be the same as or similar to the processor(s) 504 of the computing device 502 described above. The at least one processor may be part of a robot (such as a robot 514) or part of a navigation system (such as a navigation system 518). A processor other than any processor described herein may also be used to execute the method 600. The at least one processor may perform the method 600 by executing elements stored in a memory such as the memory 506. The elements stored in the memory and executed by the processor may cause the processor to execute one or more steps of a function as shown in method 600. One or more portions of a method 600 may be performed by the processor executing any of the contents of memory.

The method 600 comprises attaching a covering to a housing via a first securement feature (step 604). The housing may be the same as or similar to the housing 102, the covering may be the same as or similar to the drape or covering 106, and the first securement feature may be the same as or similar to the first securement feature 122. The housing, the covering, and an optical element such as the optical element 104 may form an assembly such as the assembly 100. The housing 102 may have a body such as the body 110 extending from a first end such as the first end 112 to the second end 114. The body may also include an opening such as the opening 118, an outer surface such as the outer surface 116, and an inner surface such as the inner surface 120. The first securement feature may comprise a first groove such as the first groove 126 disposed on the outer surface of the housing. The first groove may be configured to receive the covering and a first ring such as the first ring 128 configured to secure the covering to the first groove. In other instances, the first securement feature may comprise adhesion and the covering may be adhered to the outer surface of the housing.

The method 600 also comprises attaching an optical element to the housing via a second securement feature (step 608). The second securement feature may be the same as or similar to the second securement feature 124. In some embodiments, the optical element comprises a UV filter. The second securement feature may comprise a second groove such as the second grove 130 disposed on the inner surface of the housing. The housing 102 may also comprise a receiving surface 138 inset from a first surface such as the first surface 134 disposed at the first end of the housing. The optical element may contact the receiving surface and the second groove may receive a second ring such as the second ring 132 to secure the optical element in the housing. In other words, the optical element may be secured between the receiving surface and the second ring.

The method 600 also comprises attaching the housing to a surgical instrument (step 612). The housing may comprise a protrusion at the second end of the housing. The protrusion, in some embodiments, may be threaded. The surgical instrument may be the same as or similar to the surgical instrument 108 and may comprise a receiver for receiving the protrusion of the housing. In embodiment where the protrusion is threaded, the receiver may comprise a threaded bore for receiving the threaded protrusion, thereby coupling the housing (and thus, the optical element and the covering) to the surgical instrument.

The method 600 also comprises covering the surgical instrument with the covering (step 616). In some embodiments, the assembly may be transferred from a first location to a second location in a shipping configuration, in which the covering is folded in front of the housing. When the assembly is ready for use, the assembly may be moved to a working configuration in which the housing is coupled to the surgical instrument. The drape or covering may be unfolded and draped over the surgical instrument, thereby forming a boundary between the surgical instrument and an environment.

The present disclosure encompasses embodiments of the method 600 that comprise more or fewer steps than those described above, and/or one or more steps that are different than the steps described above.

As noted above, the present disclosure encompasses methods with fewer than all of the steps identified in FIG. 6 (and the corresponding description of the method 600), as well as methods that include additional steps beyond those identified in FIG. 6 (and the corresponding description of the method 6). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A drape assembly for a surgical instrument comprising:
a covering having an outer perimeter and an opening defining an inner perimeter, the covering configured to cover an instrument;
a housing positioned within the opening and connected to the inner perimeter, the housing configured to removably couple to the instrument; and
an optical element positioned within the housing.

2. The assembly of claim 1, wherein the housing comprises an outer surface, an inner surface, a first end, a second end opposite the first end, a first groove positioned on the outer surface between the first end and the second end, and a second groove positioned on the inner surface between the first end and the second end.

3. The assembly of claim 2, wherein the first groove receives the inner perimeter of the covering and a first ring, the first ring configured to secure the inner perimeter to the first groove, thereby connecting the housing to the inner perimeter, and wherein the second groove receives a second ring configured to secure the optical element within the housing.

4. The assembly of claim 3, wherein each of the first ring and the second ring is at least one of a c-spring, a band, or a cable tie.

5. The assembly of claim 2, wherein the second end of the housing is threaded and configured to thread into a threaded receiver of the instrument.

6. The assembly of claim 1, wherein the instrument is a camera disposed at an end of an arm, wherein the covering is configured to cover the camera and the arm.

7. The assembly of claim 1, wherein the inner perimeter is connected to the housing via adhesion.

8. The assembly of claim 1, wherein the optical element is a UV filter.

9. A housing configured to couple a surgical instrument and a covering, the housing comprising:
a body having an outer surface and an opening defining an inner surface;
a first securement feature disposed on the outer surface and configured to secure the covering to the body; and
a second securement feature disposed on the inner surface and configured to secure an optical element in the opening such that the optical element has a predetermined position relative to the covering.

10. The housing of claim 9, wherein the body comprises a first surface opposite a second surface, the first surface and the second surface perpendicular to the outer surface, wherein the body further comprises a receiving surface inset from the first surface, the receiving surface configured to receive the optical element, the receiving surface having a diameter less than a diameter of the outer surface, and wherein the second securement feature is between the receiving surface and the first surface.

11. The housing of claim 10, wherein the body comprises a threaded protrusion extending from the second surface, the threaded protrusion having a diameter less than a diameter of the outer surface, wherein the opening extends through the threaded protrusion, and wherein the threaded protrusion is configured to thread into a threaded receiver of an instrument.

12. The housing of claim 9, wherein the first securement feature extends around a perimeter of the outer surface.

13. The housing of claim 9, wherein the second securement feature extends around a perimeter of the inner surface.

14. The housing of claim 9, wherein the body is annular.

15. An assembly for a surgical instrument comprising:
a covering having an outer perimeter and an opening defining an inner perimeter, the covering configured to cover an instrument;
a housing comprising:
a body having an outer surface and an opening defining an inner surface, the inner surface and the outer surface extending from a first end to a second end;
a first securement feature configured to secure the covering to the housing;
a second securement feature configured to secure an optical element to the housing; and
the optical element positioned within the opening of the housing.

16. The assembly of claim 15, wherein the first securement feature comprises a first groove disposed on the outer surface and configured to receive a first ring and the second securement feature comprises a second groove disposed on the inner surface and configured to receive a second ring, each of the first ring and the second ring is at least one of a c-spring, a band, or a cable tie.

17. The assembly of claim 15, wherein the second end of the housing is threaded and configured to thread into a threaded receiver of the instrument.

18. The assembly of claim 15, wherein the instrument is a camera disposed at an end of an arm, wherein the covering is configured to cover the camera and the arm.

19. The assembly of claim 15, wherein the inner perimeter is connected to the housing via adhesion.

20. The assembly of claim 15, wherein the optical element is a UV filter.
